(19) United States
(12) Patent Application Publication
(10) Pub. No.: US 2009/0326232 A1
(43) Pub. Date: Dec. 31, 2009

(54) Process for the Preparation of Leukotriene Receptor Antagonist (Montelukast Sodium)

(76) Inventors: Uttam Kumar Ray, Andhra Pradesh (IN); Sreenivasulu Boju, Andhra Pradesh (IN); Sreenivasa Rao Pathuri, Andhra Pradesh (IN); Sivakumaran Meenakshisunderam, Andhra Pradesh (IN)

Correspondence Address: Jay Akhave, 2058 N Mill Avenue #612, Claremont, CA, 91711, US

(21) Appl. No.: 12/308,833

(22) PCT Filed: Jun. 25, 2007
(86) PCT No.: PCT/IB2007/001870

§ 371 Date: Dec. 23, 2008

(30) Foreign Application Priority Data
Jun. 26, 2006 (IN) 1084/CHE/2006
Jun. 26, 2006 (IN) 1085/CHE/2006

(51) Int. Cl. C07D 215/14

(52) U.S. Cl. 546/174

(57) ABSTRACT

The present invention relates to an improved process for the preparation of [R-(E)]-1-[[[1-[3-[2-(7-chloroquinolin-2-yl)ethenyl]phenyl]-3-[2-(1-hydroxy-1-methylethyl)phenyl]propyl]thio]methyl]-cyclopropaneacetic acid, monosodium salt of Formula (I).

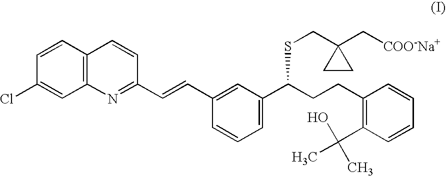

### FIELD OF THE INVENTION

The present invention relates to an improved process for the preparation of [R-(E)]-1-[[[1-[3-[2-(7-chloroquinolin-2-yl)ethenyl]phenyl]-3-[2-(1-hydroxy-1-methylethyl)phenyl]-propyl]thio]methyl]cyclopropaneacetic acid, monosodium salt of Formula (I).

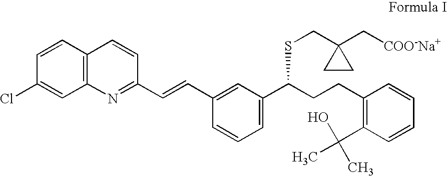

### BACKGROUND OF THE INVENTION

[R-(E)]-1-[[[1-[3-[2-(7-Chloroquinolin-2-yl)ethenyl]phenyl]-3-[2-(1-hydroxy-1-methylethyl)phenyl]propyl]thio]methyl]cyclopropaneacetic acid, monosodium salt (I) is generically known as Montelukast sodium. Montelukast and its pharmaceutically acceptable salts are selective Leukotriene receptor antagonists, which inhibit the cysteinyl leukotriene CysLT₁ receptor. Montelukast sodium is used in the treatment of asthma and allergic rhinitis.

Merck & Co., has disclosed Montelukast and its derivatives for the first time in U.S. Pat. No. 5,565,473. U.S. Pat. No. 5,565,473 also discloses the process of preparing Montelukast sodium by condensing 2-(2-(2-(3(S)-(3-(2-(7-chloroquinolin-2-yl)ethenyl)phenyl)-3-(methanesulfonyloxy)methylethyl)phenyl)-2-propoxy)tetrahydropyran (II) with methyl-1-(acetylthiomethyl)cyclopropane acetate (III) in presence of cesium carbonate to produce methyl-4-((1(R)-(3-(2-(7-chloroquinolin-2-yl)ethenyl)phenyl)-3-(2-(2-(2-tetrahydropyranyloxy)-2-methylethyl)phenyl)propyl)thio)cyclopropyl acetate (IV), which is de-protected in presence of pyridinium-p-toluenesulfonate and further converted to Montelukast sodium (I) in presence of sodium base in a mixture of solvents selected from methanol and tetrahydrofuran.

The process is shown in Scheme-I below:

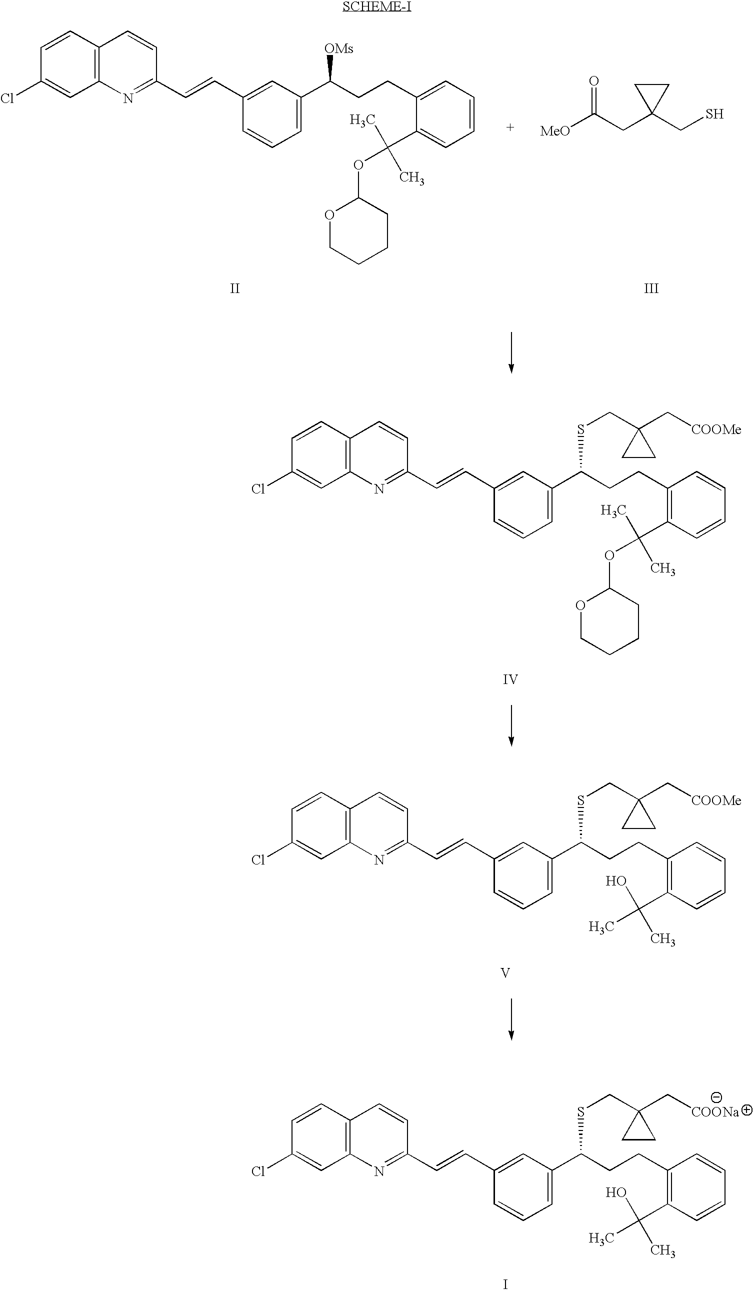

The above process is not particularly suitable for large-scale production, because it requires tedious chromatographic purification of intermediates and final product, which finally results in the low yield of Montelukast sodium.

U.S. Pat. No. 5,614,632 discloses a process for the preparation of Montelukast sodium through Montelukast DCHA salt (IX). The process comprises, condensing 2-(2-(2-(3(S)-(3-(2-(7-chloroquinolin-2-yl)ethenyl)phenyl)-3-(methanesulfonyloxy)methylethyl)phenyl)-2-propanol (VI) with anion of 1-(mercaptomethyl)cyclopropaneacetic acid (VII) [Anion of 1-(mercaptomethyl)cyclopropaneacetic acid is produced by the reaction of 1-(mercaptomethyl)cyclopropaneacetic acid with n-butyl lithium] to produce crude Montelukast and then converted into dicyclohexylamine salt. Montelukast dicyclohexylamine salt is further converted to Montelukast sodium by treating with sodium hydroxide.

The process is shown in Scheme-II below:

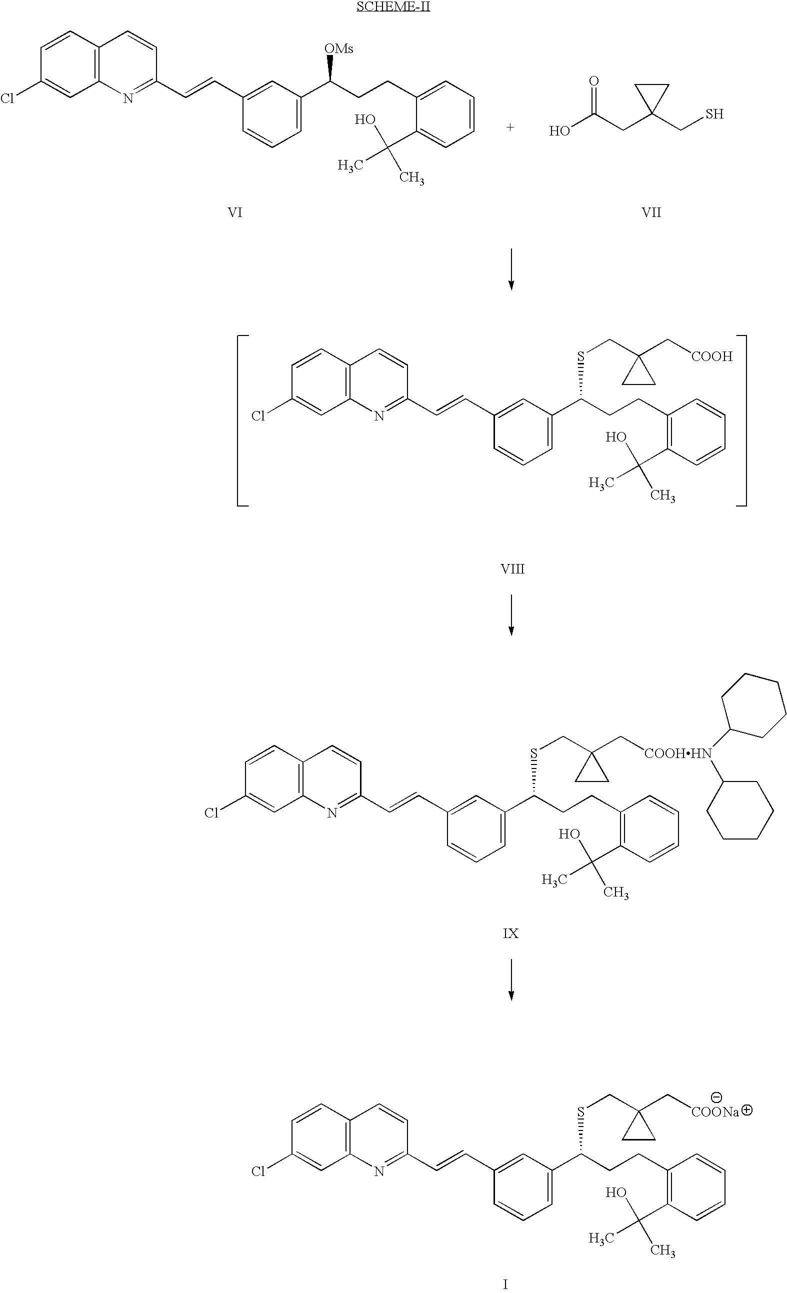

The above process also suffers with major disadvantage of low yield and low purity of Montelukast. The reason for such low yield is that during the condensation of 2-(2-(2-(3(S)-(3-(2-(7-chloroquinolin-2-yl)ethenyl)phenyl)-3-(methanesulfonyloxy)methylethyl)-phenyl)-2-propanol (VI) with 1-(mercaptomethyl)cyclopropaneacetic acid (VII) in presence of strong base n-butyl lithium undesired impurities are generated. The removal of these impurities is often proved to be difficult and requires separate crystallization, which finally results in the low yield of Montelukast sodium (I).

The present invention relates to the use of mild bases in condensation of 2-(2-(2-(3(S)-(3-(2-(7-chloroquinolin-2-yl)ethenyl)phenyl)-3-(methanesulfonyloxy)methylethyl)phenyl)-2-propanol (VI) with 1-(mercaptomethyl)cyclopropaneacetic acid methyl ester (III).

The present invention also relates to a process for the preparation of Montelukast, where 2-(2-(2-(3(S)-(3-(2-(7-chloroquinolin-2-yl)ethenyl)phenyl)-3-(methanesulfonyloxy)-methylethyl)phenyl)-2-propoxy)tetrahydropyran (II) is condensed with 1-(mercaptomethyl)cyclopropaneacetic acid (VII) and the resulting protected intermediate used as such to produce Montelukast sodium of high purity and yield.

### OBJECTIVE OF THE INVENTION

The main objective of the present invention is to provide a simple and effective process for the preparation of Montelukast sodium of high purity and yield on a commercial scale.

### SUMMARY OF THE INVENTION

The present invention relates to an improved process for the preparation of [R-(E)]-1-[[[1-[3-[2-(7-chloroquinolin-2-yl)ethenyl]phenyl]-3-[2-(1-hydroxy-1-methylethyl)phenyl]-propyl]thio]methyl]cyclopropaneacetic acid, monosodium salt of Formula (I) which comprises;
i) condensing Mesyl derivative of formula (X)

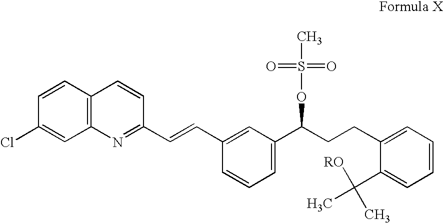

wherein, R is H or hydroxyl protecting group with a compound of formula (XI)

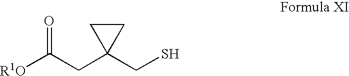

wherein, R¹ is H or alkyl group, preferably methyl or ethyl in presence of base in a solvent to produce Montelukast acid derivative (XII);

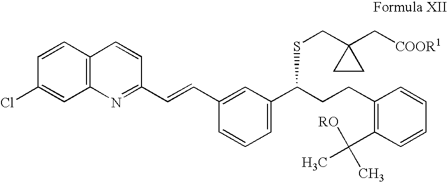

wherein, R is H or hydroxyl protecting group; R¹ is H or alkyl group, preferably methyl or ethyl
ii) converting Montelukast acid derivative of Formula (XII) to dicyclohexylamine salt of 1-(((1-(R)-(3-(2-(7-chloroquinolin-2-yl)ethenyl)phenyl)-3-(2-(1-hydroxy-1-methyl-ethyl)phenyl)propyl)thio)methyl)cyclopropaneacetic acid of Formula (IX) (Montelukast DCHA salt);

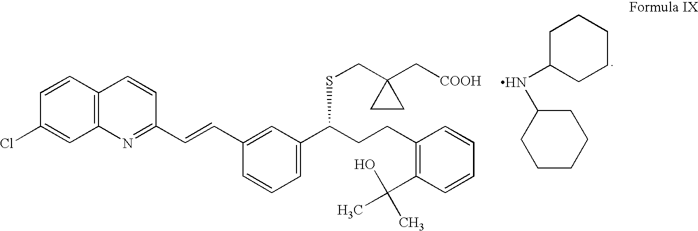

iii) treating the Montelukast DCHA salt (IX) with an acid in a solvent to give Montelukast free acid, which on further treatment with sodium ion source in a solvent produces [R-(E)]-1-[[[1-[3-[2-(7-chloroquinolin-2-yl)ethenyl]phenyl]-3-[2-(1-hydroxy-1-methyl-ethyl)phenyl]propyl]thio]methyl]cyclopropaneacetic acid, monosodium of Formula (I) (Montelukast sodium).
   with a proviso (a) when R is hydrogen, R¹ is hydrogen or alkyl group, preferably methyl or ethyl, the base used in step (i) is selected from alkali or alkaline earth metal carbonates such as cesium carbonate, potassium carbonate, sodium carbonate, lithium carbonate, rubidium carbonate and, when R is hydroxyl protecting group such as tetrahydropyran, R¹ is hydrogen or alkyl group, preferably methyl or ethyl, the base used is alkyl lithium selected from n-butyl lithium, methyl lithium and isopropyl lithium;
   (b) step (ii) is carried out either in presence of base when R is hydrogen or R¹ is methyl and in presence of catalyst when R is hydroxyl protecting group and R¹ is hydrogen.

### DETAILED DESCRIPTION OF THE INVENTION

The present invention relates to an improved process for the preparation of [R-(E)]-1-[[[1-[3-[2-(7-chloroquinolin-2-yl)ethenyl]phenyl]-3-[2-(1-hydroxy-1-methylethyl)phenyl]-propyl]thio]methyl]cyclopropaneacetic acid, monosodium (I) (Montelukast sodium).

Alkyl group as used herein refers to methyl, ethyl, propyl.

The mesyl derivative of Formula (X), wherein R is hydrogen is reacted with the compound of Formula (XI) is in presence of a base selected from cesium carbonate, potassium carbonate, sodium carbonate, lithium carbonate, rubidium carbonate and a solvent selected from acetonitrile, acetone and THF, preferably in acetonitrile. The reaction is carried out at a temperature of about 0° C. to about −20° C., preferably 0° C. to −5° C. under nitrogen atmosphere. The reaction mass is stirred for about 5 to 10 h, preferably 5 h. After completion of the reaction, the reaction mass is poured in a mixture of aqueous sodium chloride and solvent selected from ethyl acetate, methyl acetate and isopropyl acetate. Montelukast acid derivative (XII) is finally isolated from the organic layer by evaporating the solvent.

In another embodiment, the compound of Formula (XI), wherein R¹ is hydrogen, is treated with alkyl lithium in presence of solvent selected from tetrahydrofuran, hexanes, toluene, preferably in tetrahydrofuran at a temperature of about −10° C. to about −50° C., preferably −15 to −20° C. under nitrogen atmosphere to produce dilithium anion of compound of Formula (XI). The alkyl lithium is selected from n-butyl lithium, methyl lithium and isopropyl lithium. Dilithium anion of compound of Formula (XI) is reacted with mesyl derivative of formula (X), wherein R is a hydroxyl protecting group i.e., tetrahydropyranyl in a solution of tetrahydrofuran, hexanes or toluene, preferably in tetrahydrofuran. The reaction is carried out at a temperature of about 0° C. to −20° C., preferably −5° C. to about −10° C. The reaction mass is stirred for about 5 to 10 h, preferably 7-8 h. After completion of the reaction the reaction mass is poured in to 1:1 v/v mixture of aqueous sodium chloride and a suitable solvent selected from ethyl acetate, methylene chloride, toluene, 1,2-dichloroethane. Montelukast acid derivative (XII) is finally isolated from the organic layer by evaporating the solvent.

Montelukast acid derivative (XII), wherein, R is hydrogen and R¹ is alkyl group, is treated with a base selected from NaOH, LiOH and KOH in a solvent selected from methanol, tetrahydrofuran, dioxane or mixtures thereof, preferably 3:1 mixture of methanol and tetrahydrofuran. The reaction is carried out at room temperature for about 2 days. After the completion of the reaction, solvent is evaporated under reduced pressure at 35° C. to 40° C. and the pH is adjusted to 5 with acetic acid and ammonium chloride. The residue containing crude Montelukast is suspended in a solvent selected from toluene, xylene, preferably toluene, is treated with N,N-dicyclohexylamine. The resulting mass is treated with carbon and the filtrate is stirred for 2 to 5 h, preferably 3 h at a temperature of about 10 to 30° C. and solvent selected from n-heptane, hexane and n-pentane or mixtures thereof is added to the above filtrate slowly for about 1 to 2 h and the resulting mass is stirred for 10 to 15 h to produce dicyclohexyl amine salt of 1-(((1-(R)-(3-(2-(7-chloroquinolin-2-yl)ethenyl)phenyl)-3-(2-(1-hydroxy-1-methylethyl)phenyl)propyl)thio)methyl)-cyclopropaneacetic acid (IX) (Montelukast DCHA salt).

In another embodiment, Montelukast acid derivative (XII), wherein, R is hydroxyl protecting group and R¹ is hydrogen is treated with a catalyst selected from pyridinium-para-toluenesulfonate, pyridinium-ortho-toluenesulfonate, pyridinium-benzenesulfonate, pyridinium-methanesulfonate, preferably pyridinium-para-toluenesulfonate in a solvent selected from methanol, ethanol, isopropanol, n-butanol, tetrahydrofuran, dioxane or mixtures thereof, preferably 3:1 mixture of methanol and tetrahydrofuran. The reaction is carried out at a temperature of about 40° C. to 80° C., preferably at about 55 to 60° C. for about 8 to 15 h, preferably 10 to 12 h. The residue containing crude Montelukast is suspended in a solvent selected from toluene, xylene, preferably toluene and treated with N,N-dicyclohexylamine. The resulting mass is treated with carbon and the filtrate is stirred for 2 to 5 h, preferably 3 h at a temperature of about 10 to 30° C. and solvent selected from n-heptane, hexane and n-pentane or mixtures thereof is added slowly for about 1 to 2 h. The resulting mass is stirred for 10 to 15 h to produce dicyclohexylamine salt of 1-(((1-(R)-(3-(2-(7-chloroquinolin-2-yl)ethenyl)phenyl)-3-(2-(1-hydroxy-1-methylethyl)phenyl)-propyl)thio)methyl)cyclopropaneacetic acid (IX) (Montelukast DCHA salt).

Montelukast DCHA salt (IX) is acidified with acid selected from organic or inorganic acid, preferably an organic acid such as acetic acid, tartaric acid, oxalic acid in a solvent selected from water, organic solvent such as toluene, xylene or mixtures thereof. The organic layer is separated and evaporated completely under reduced pressure and the residue containing Montelukast free acid is dissolved in a solvent selected from methylene chloride or ethylene dichloride and precipitated by adding antisolvent selected from hexane, n-heptane and n-pentane. Filtration affords Montelukast free acid as a solid. Further Montelukast free acid can be converted to Montelukast sodium by conventional methods such as freeze drying in presence of sodium ion source selected from sodium hydroxide in a solvent selected from water.

The following examples illustrate the nature of the invention and are provided for illustrative purposes only and should not be construed to limit the scope of the invention.

### REFERENCE EXAMPLE

### Step-1

### Methyl-4-((1(R)-(3-(2-(7-chloroquinolin-2-yl)ethenyl)phenyl)-3-(2-(2-(2-tetrahydropyranyl-oxy)-2-methylethyl)phenyl)propyl)thio)cyclopropylacetate

### Protected Montelukast Methyl Ester

1-(Mercaptomethyl)cyclopropane methyl acetate (0.96 g, 6 mmol) was dissolved in acetonitrile (10 ml) at 27-30° C. under nitrogen atmosphere. The solution was then added to a suspension of 2-(2-(2-(3(S)-(3-(2-(7-chloro-2-quinolinyl)ethenyl)phenyl)-3-(methanesulfonyloxy)methylethyl)phenyl)-2-propoxy)tetrahydropyran (mesyl derivative) (2.5 g, 4.0 mmol) and potassium carbonate (1.36 g, 9.8 mmol) in acetonitrile (10 ml) at 0° C. to −5° C. under nitrogen atmosphere over a period of 1 h. The reaction mixture was warmed to 27-30° C. and stirred at 27-30° C. for 36 h. The progress of the reaction was monitored by TLC.

Water (20 ml) was added to the above reaction mass and extracted with ethyl acetate (50 ml). The ethyl acetate layer was washed with brine, dried over Na₂SO₄ and then concentrated to obtain methyl-4-((1(R)-(3-(2-(7-chloroquinolin-2-yl)ethenyl)phenyl)-3-(2-(2-(2-tetrahydropyranyl-oxy)-2-methylethyl)phenyl)propyl)thio)cyclopropylacetate (Protected Montelukast methyl ester).

Yield 2.0 g (72.5%).

### Example-1

### Step I

### Preparation of 2-(2-(3(S)-(3-(2-(7-chloroquinolin-2-yl)ethenyl)phenyl)-3-(methanesulfonyl-oxy)methylethyl)phenyl)-2-propanol (mesyl derivative)

2-(2-(3(S)-(3-(2-(7-Chloroquinolin-2-yl)ethenyl)phenyl)-3-(hydroxypropyl)phenyl)-2-propanol (Diol derivative) (20 g, 0.0437 mol) was dissolved in toluene (60 ml) and acetonitrile (160 ml) mixture under nitrogen atmosphere at room temperature. Diisopropylethylamine (8.5 g, 0.0656 mol) was added to the reaction mixture and the temperature was lowered to −35° C. to −30° C. Methanesulfonyl chloride (7.0 g, 0.0612 mol) was added slowly over a period of 1 h at −30° C. to −25° C. and the reaction mixture was stirred at −27° C. to −25° C. for 2 h. Thereafter the temperature was lowered to −35° C. to −30° C. and stirred for another 2 h. After completion of the reaction the precipitated product was filtered the product under nitrogen blanket at −30° C. and washed with cold acetonitrile (80 ml, −30° C.) followed by hexanes (2×80 ml, 0° C.). Product was dried at 5° C. under positive pressure of nitrogen to get 21 g of 2-(2-(3(S)-(3-(2-(7-chloroquinolin-2-yl)ethenyl)phenyl)-3-(methanesulfonyloxy)methylethyl)phenyl)-2-propanol (mesyl derivative).

Yield (87.74%)

### Step II

### Preparation of 1-(((1-(R)-(3-(2-(7-chloroquinolin-2-yl)ethenyl)phenyl)-3-(2-(1-hydroxy-1-methylethyl)phenyl)propyl)thio)methyl)cyclopropaneacetic acid methyl ester

### Montelukast Methyl Ester

Mesyl derivative (5 g, 0.00934 mol) was added to a suspension of dried cesium carbonate (6.1 g, 0.0187 mol) in acetonitrile (15 ml) at −3° C. under nitrogen atmosphere. The resulting suspension was stirred at −7° C. to −3° C. for 15 min. 1-(Mercaptomethyl)cyclopropaneacetic acid methyl ester (1.5 g, 0.009375 mol) in acetonitrile (15 ml) at −7° C. was added to the above solution in one lot. The temperature was slowly raised to room temperature and stirred for 5 h at this temperature while monitoring the progress of the reaction by TLC.

After completion of the reaction, DM water (50 ml) was added to mass below 110° C. and the product was extracted with ethyl acetate. The organic layer was washed with brine solution and dried over anhydrous sodium sulfate. Concentrating organic layer under reduced pressure at 40° C. gave 5 g residue of 1-(((1-(R)-(3-(2-(7-chloroquinolin-2-yl)ethenyl)phenyl)-3-(2-(1-hydroxy)methylethyl)phenyl)propyl)thio)methyl)cyclopropaneacetic acid methyl ester (Montelukast methyl ester).

Yield (89.32%).

### Step III

### Preparation of 1-(((1-(R)-(3-(2-(7-chloroquinolin-2-yl)ethenyl)phenyl)-3-(2-(1-hydroxy-1-methylethyl)phenyl)propyl)thio)methyl)cyclopropaneacetic acid

### Montelukast Crude

Montelukast methyl ester (5.0 g, 0.00834 mol) was dissolved in a mixture of methanol: tetrahydrofuran (3:1, 40 ml) at room temperature. The solution was cooled to 0° C. and 1M sodium hydroxide solution (20 ml, 0.02 mol) was added and stirred the reaction mass at room temperature for 2 days. Most of the methanol and THF was evaporated under reduced pressure at 35-40° C. The pH of the reaction mass was adjusted to 5 with acetic acid, ammonium chloride solution and extracted with ethyl acetate. The organic layer was washed with 20% w/v sodium chloride solution, dried over anhydrous sodium sulfate and concentrated under reduced pressure at 40-45° C. to get 3.2 g solid or viscous mass of 1-(((1-(R)-(3-(2-(7-chloroquinolin-2-yl)ethenyl)phenyl)-3-(2-(1-hydroxy-1-methylethyl)-phenyl)propyl)thio)methyl)cyclopropaneacetic acid (Montelukast crude). Yield (65.57%)

### Step IV

### Preparation of Dicyclohexylamine salt of 1-(((1-(R)-(3-(2-(7-chloroquinolin-2-yl)ethenyl)phenyl)-3-(2-(1-hydroxy-1-methylethyl)phenyl)propyl)thio)methyl)cyclopropaneacetic acid

The crude Montelukast (3.2 g, 5.47 mmol) obtained as above was suspended in toluene (24 ml) and the suspension was stirred for 30 min under nitrogen atmosphere to get a clear solution. Dicyclohexylamine (1.1 g, 6 mmol) was added to the clear solution and the reaction mass was stirred for 15 min at 20-25° C. Carbon (0.32 g) was added and stirred the mass for 1 h at 20-25° C. Thereafter the mass was filtered through hyflo and washed the hyflo bed with toluene (6.5 ml). The filtrate was diluted with 16 ml of toluene and seeded with dicyclohexylamine salt of Montelukast (25 mg). The solution was stirred for 3 h at 20-25° C. and n-heptane (23 ml) was added very slowly over a period of 2 h at 20-25° C., to result in thick white slurry. The slurry was filtered and dried to yield Montelukast dicyclohexylamine salt (2.5 g).

Purity 97.0% by HPLC.

Yield (59.67%).

### Step V

### Preparation of 1-(((1-(R)-(3-(2-(7-chloroquinolin-2-yl)ethenyl)phenyl)-3-(2-(1-hydroxy-1-methylethyl)phenyl)propyl)thio)methyl)cyclopropaneacetic acid

### Montelukast

Montelukast DCHA salt (2.5 g, 3.26 mmol) was suspended into a mixture of toluene (50 ml) and DM water (37.5 ml) and the resulting mixture was treated with acetic acid until a clear solution resulted. The organic layer was separated and the toluene was evaporated completely under reduced pressure to obtain yellowish residue. Methylene chloride (17.5 ml) was added to the residue and stirred at 35° C. to get the clear solution and hexane was added slowly in 1 h. The Montelukast obtained was filtered and washed with a mixed solution of methylene chloride and hexane (1:3, 12 ml) and dried.

Yield: 1.65 g (86.39%).

Purity 98.12% by HPLC.

### Example-2

### Step I

### Preparation of 1-(((1-(R)-(3-(2-(7-chloroquinolin-2-yl)ethenyl)phenyl)-3-(2-(1-hydroxy-1-methylethyl)phenyl)propyl)thio)methyl)cyclopropaneacetic acid methyl ester

### Montelukast Methyl Ester

2-(2-(3(S)-(3-(2-(7-Chloroquinolin-2-yl)ethenyl)phenyl)-3-(methanesulfonyloxy)-methylethyl)phenyl)-2-propanol (mesyl derivative) (9 g, 16.8 mmol) was stirred with potassium carbonate (5.8 g, 42 mmol) in acetonitrile (30 ml) at −5° C. 1-(Mercaptomethyl)cyclopropaneacetic acid methyl ester (4 g, 25 mmol) in acetonitrile (30 ml) was added in 15 min at −5° C. to 0° C. The reaction mass temperature was slowly raised to 20° C. and stirred at 20-25° C. for 16 h. TLC showed complete disappearance of starting material.

After completion of the reaction water was added below 10° C. and extracted with ethyl acetate. The organic layer was washed with brine solution, dried over anhydrous sodium sulfate and concentrated under reduced pressure to get 10 g residue of 1-(((1-(R)-(3-(2-(7-chloroquinolin-2-yl)ethenyl)phenyl)-3-(2-(1-hydroxy)methylethyl)phenyl)propyl)thio)-methyl)cyclopropaneacetic acid methyl ester (Montelukast methyl ester).

Yield (99.3%)

### Step II

### Preparation of 1-(((1-(R)-(3-(2-(7-chloroquinolin-2-yl)ethenyl)phenyl)-3-(2-(1-hydroxy-1-methylethyl)phenyl)propyl)thio)methyl)cyclopropaneacetic acid

### Montelukast Crude

Montelukast methyl ester (10 g, 16.8 mmol) was dissolved in a mixture of methanol:THF (3:1, 63 ml) and cooled the solution to below 0° C. To this reaction mixture 1M sodium hydroxide solution (40.3 ml, 40.3 mmol) was added at 0° C. in 15 min and the reaction mass was stirred at room temperature for 2 days. Most of the methanol and THF was evaporated under reduced pressure at 40° C. and the pH was adjusted to 5 with acetic acid. Ammonium chloride solution was added to the reaction mass and extracted with ethyl acetate. The organic layer was dried over anhydrous sodium sulfate and concentrated under reduced pressure at 35-40° C. to get 9.0 g residue of 1-(((1-(R)-(3-(2-(7-chloroquinolin-2-yl)ethenyl)phenyl)-3-(2-(1-hydroxy-1-methylethyl)phenyl)propyl)thio)methyl)-cyclopropaneacetic acid (Montelukast crude).

### Step III

### Preparation of dicyclohexylamine salt of 1-(((1-(R)-(3-(2-(7-chloroquinolin-2-yl)ethenyl)phenyl)-3-(2-(1-hydroxy-1-methylethyl)phenyl)propyl)thio)methyl)cyclopropaneacetic acid

The crude Montelukast (3.2 g, 5.47 mmol) obtained in step II was suspended in toluene (24 ml) and stirred for 30 min under nitrogen atmosphere to get a clear solution. Dicyclohexylamine (1.1 g, 6 mmol) was added to the clear solution and the reaction mass was stirred for 15 min at 20-25° C. Carbon (0.32 g) was added and stirred the mass for 1 h at 20-25° C. and mass was filtered through hyflo and washed the hyflo bed with toluene (6.5 ml). The filtrate was diluted with 16 ml of toluene and seeded with dicyclohexylamine salt of Montelukast (25 mg). The solution was stirred for 3 h at 20-25° C. and n-heptane (23 ml) was added very slowly over a period of 2 h at 20-25° C., to obtain a thick white slurry. The slurry containing Montelukast dicyclohexyl amine salt was filtered and dried (2.5 g). Purity 97.0% by HPLC.

Yield (59.67%)

### Step IV

### Preparation of 1-(((1-(R)-(3-(2-(7-chloroquinolin-2-yl)ethenyl)phenyl)-3-(2-(1-hydroxy-1-methylethyl)phenyl)propyl)thio)methyl)cyclopropaneacetic acid

### Montelukast

Montelukast DCHA salt (2.5 g, 3.26 mmol) was suspended in a mixture of toluene (50 ml) and DM water (37.5 ml) and the resulting mixture was treated with acetic acid until a clear solution was obtained. The organic layer was separated and the toluene was evaporated completely under reduced pressure to obtain yellowish residue. Methylene chloride (17.5 ml) was added to the residue and stirred at 35° C. to get a clear solution and hexane (52.5 ml) was added slowly in 1 h. The Montelukast obtained was filtered and washed with a mixture of methylene chloride and hexane (1:3, 12 ml) and dried.

Yield: 1.65 g (86.39%).

Purity 98.21% by HPLC.

### Example-3

### Step I

### Preparation of 2-(2-(3(S)-(3-(2-(7-chloroquinolin-2-yl)ethenyl)phenyl)-3-(methanesulfonyl-oxy)methylethyl)phenyl)-2-propoxy)tetrahydropyran

### Mesyl Derivative

1-(S)-(3-(2-(7-Chloro-2-quinolinyl)ethenyl)phenyl)-3-(2-(2-((tetrahydropyran-2-yl)oxy)-2-propyl)-phenyl-1-propanol (18 g, 33 mmol) was dissolved in methylene chloride (300 ml) at 25-30° C., triethylamine (5 g, 50 mmol) was added and cooled the solution to −50° C. Methanesulfonyl chloride (4.94 g, 43 mmol) was added at −50° C. to −40° C. The resulting solution was stirred at −50° C. to −40° C. for 1 h. After completion of reaction, the mixture was diluted with saturated sodium bicarbonate and extracted with CH₂Cl₂. The methylene chloride layer was dried over sodium sulfate and evaporated under reduced pressure to obtain mesyl derivative as oily mass (19 g).

### Step II

### Preparation of 1-(((1-(R)-(3-(2-(7-chloroquinolin-2-yl)ethenyl)phenyl)-3-(2-(2-(2-tetrahydropyranyl)oxy)methylethyl)phenyl)propyl)thio)methyl)cyclopropaneacetate

### Tetrahydropyranyl Montelukast

1-(Mercaptomethyl)cyclopropane acetic acid (5.10 g, 35 mmol) was dissolved in tetrahydrofuran (150 ml) at 25-30° C. and cooled the solution to −15° C. n-Butyl lithium (26.46 g. 413 mmol) was added under nitrogen atmosphere and stirred the resulting slurry at −5° C. for 30 min. Thereafter mesyl derivative produced in step I (18 g, 29 mmol) was dissolved in tetrahydrofuran (80 ml) at 0° C. under nitrogen and added to the slurry at −5° C. to −10° C. slowly. The reaction mass was stirred at −5° C. to 0° C. for 8 h and progress of the reaction was monitored by TLC.

After completion of reaction, the reaction mass was poured into 1:1 v/v mixture of ethyl acetate and 10% w/v aqueous sodium chloride solution. The organic phase was washed with 0.5 M tartaric acid and with DM water. The solvent was removed under reduced pressure to obtain tetrahydropyranyl Montelukast as an oily compound (19.80 g).

### Step III

### Preparation of 1-(((1-(R)-(3-(2-(7-chloroquinolin-2-yl)ethenyl)phenyl)-3-(2-(1-hydroxy-1-methylethyl)phenyl)propyl)thio)methyl)cyclopropaneacetic acid

### Crude Montelukast

A solution of Step II containing tetrahydropyranyl Montelukast (19 g, 28 mmol) was treated with pyridinium p-toluene sulfonate (21.36 g, 85 mmol) in a 3:1 mixture of methanol: tetrahydrofuran (160 ml). The reaction mixture was warmed to 60° C. and stirred for 12 h, the progress of the reaction was monitored by TLC. After completion of the reaction most of the methanol was evaporated and an aqueous solution of ammonium chloride was added. The crude Montelukast was extracted with ethyl acetate, washed with sodium chloride and dried over Na₂SO₄. Ethyl acetate was evaporated to obtain the title compound (16 g).

### Step IV

### Preparation of dicyclohexylamine salt of 1-(((1-(R)-(3-(2-(7-chloroquinolin-2-yl)ethenyl)phenyl)-3-(2-(1-hydroxy-1-methylethyl)phenyl)propyl)thio)methyl)cyclopropaneacetic acid

The crude Montelukast (10 g, 17 mmol) was suspended in toluene (75 ml) and the suspension was stirred for 30 min under nitrogen atmosphere to get a clear solution. Dicyclohexylamine (3.38 g, 19 mmol) was added to the clear solution and the reaction mass was stirred for 15 min at 20-25° C. Carbon (1 g) was added and stirred the mass for 1 h at 20-25° C. Thereafter, carbon was filtered off through hyflo and washed the hyflo bed with toluene (20 ml). Toluene (50 ml) was added to the reaction mixture and seeded with DCHA salt of Montelukast (50 mg). The solution was stirred for 3 h at 20-25° C. and n-heptane (70 ml) was added very slowly over a period of 2 h at 20-25° C., to yield a thick white slurry. The slurry containing Montelukast dicyclohexyl amine salt was filtered and dried (6 g).

### Step V

### Preparation of 1-(((1-(R)-(3-(2-(7-chloroquinolin-2-yl)ethenyl)phenyl)-3-(2-(1-hydroxy-1-methylethyl)phenyl)propyl)thio)methyl)cyclopropaneacetic acid

### Montelukast

Montelukast DCHA salt (5 g, 6.5 mmol) was suspended in a mixture of toluene (100 ml) and DM water (75 ml) and the resulting solution was treated with acetic acid until a clear solution was obtained. The organic layer was separated and the toluene was evaporated completely under reduced pressure to obtain yellowish residue containing Montelukast. Methylene chloride (35 ml) was added to the residue and stirred at 35° C. to get a clear solution and hexane was added slowly in 1 h. The Montelukast obtained was filtered and washed with hexane (25 ml) and dried. Yield: 3.40 g.

We claim:

1) An improved process for the preparation of [R-(E)]-1-[[[1-[3-[2-(7-chloroquinolin-2-yl)ethenyl]phenyl]-3-[2-(1-hydroxy-1-methylethyl)phenyl]propyl]thio]methyl]cyclopropaneacetic acid, monosodium salt of Formula I which comprises;
i) condensing Mesyl derivative of formula (X) 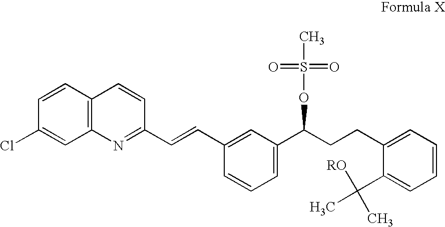
wherein, R is H or hydroxyl protecting group with a compound of formula (XI) 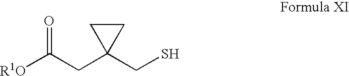
wherein, R¹ is H or alkyl group, preferably methyl or ethyl in presence of base in a solvent to produce Montelukast acid derivative of Formula (XII); 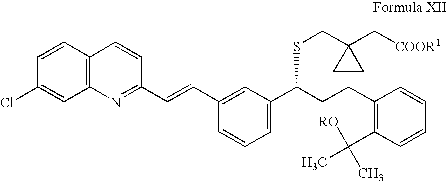
wherein, R is H or hydroxyl protecting group; R¹ is H or alkyl group, preferably methyl or ethyl
ii) converting Montelukast acid derivative of Formula (XII) to dicyclohexylamine salt of 1-(((1-(R)-(3-(2-(7-chloroquinolin-2-yl)ethenyl)phenyl)-3-(2-(1-hydroxy-1-methylethyl)phenyl)propyl)thio)methyl)cyclopropaneacetic acid of Formula (IX) (Montelukast DCHA salt); 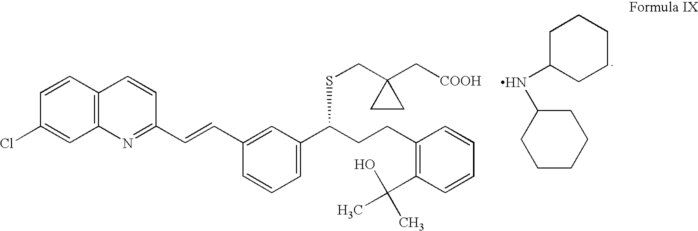
iii) treating the Montelukast DCHA salt of Formula (IX) with an acid in a solvent to give Montelukast free acid, which on further treatment with sodium ion source in a solvent produces [R-(E)]-1-[[[1-[3-[2-(7-chloroquinolin-2-yl)ethenyl]phenyl]-3-[2-(1-hydroxy-1-methylethyl)phenyl]propyl]thio]methyl]-cyclopropaneacetic acid, monosodium (I) (Montelukast sodium).
with a proviso (a) when R is hydrogen, R¹ is hydrogen or alkyl group, preferably methyl or ethyl, the base used in step (i) is selected from alkali or alkaline earth metal carbonates and, when R is hydroxyl protecting group such as tetrahydropyranyl, R¹ is hydrogen or alkyl group, preferably methyl or ethyl, the base used is alkyl lithium; (b) step (ii) is carried out in presence of base when R is hydrogen and R¹ is methyl and in presence of catalyst when R is hydroxyl protecting group and R¹ is hydrogen.

2) A process according to the claim 1, wherein alkali or alkaline earth metal carbonates are selected form the group of cesium carbonate, potassium carbonate, sodium carbonate, lithium carbonate and rubidium carbonate.

3) A process according to claim 1, wherein alkyl lithium is selected form the group of n-butyl lithium, methyl lithium and isopropyl lithium.

4) A process according to the claim 1, wherein the condensation step is carried out in a solvent selected from acetonitrile, acetone, THF, hexanes and toluene or mixtures thereof.

5) A process according to the claim 1, wherein the base used in step (ii) is selected from NaOH, LiOH, KOH or mixtures thereof.

6) A process according to claim 9, wherein the catalyst used in step (ii) is selected from pyridinium-para-toluenesulfonate, pyridinium-ortho-toluenesulfonate, pyridinium-benzenesulfonate, pyridinium-methanesulfonate, preferably pyridinium-para-toluenesulfonate.

7) A process according to the claim 1, wherein the solvent used in step (ii) is selected from methanol, ethanol, isopropanol, n-butanol, tetrahydrofuran, dioxane or mixtures thereof.

8) A process according to the claim 1, wherein the acid used in step (iii) is selected from organic acid such as acetic acid, tartaric acid, oxalic acid or inorganic acid such as HCl or HBr.

9) A process according to the claim 1, wherein the solvent used in step (iii) is selected from water, toluene, xylene or mixtures thereof.

10) A process according to the claim 1, wherein the sodium ion source is selected from sodium hydroxide, sodium ethoxide, sodium methoxide, sodium acetate, sodium propionate, sodium benzoate and sodium 2-ethyl hexanoate.
